(19) United States
(12) Patent Application Publication
(10) Pub. No.: US 2002/0019576 A1
(43) Pub. Date: Feb. 14, 2002

(54) Process and facility for the separation of acid and/or alkaline constituents from hydrocarbons

(76) Inventors: Gerhard Emmrich, Essen (DE); Frank Ennenbach, Eggenstein (DE); Uwe Ranke, Essen (DE); Helmut Gehrke, Beckum-Vellern (DE)

Correspondence Address: MARSHALL & MELHORN, LLC, Phillip S. Oberlin, 8th Floor, Four SeaGate, Toledo, OH, 43604, US

(21) Appl. No.: 09/915,699

(22) Filed: Jul. 26, 2001

(30) Foreign Application Priority Data
Aug. 5, 2000 (DE) 100 38 320.3

(51) Int. Cl. C07C 7/00

(52) U.S. Cl. 585/866; 585/804; 585/809; 585/803

(57) ABSTRACT

The invention relates to a process for purifying hydrocarbon vapor consisting of at least one aromatic or one olefinic or one paraffinic compound or of a mixture thereof, this hydrocarbon vapor entraining impurities of acid or alkaline nature which consist of at least one water-soluble organic and/or inorganic substance and it also relates to a facility for performing such a process.

The product obtained by the process according to the invention is characterized by an extremely high purity as regards impurities in the form of organic and inorganic compounds of the elements sulfur, nitrogen, oxygen and chlorine. To this end, an aqueous solution 7 is dispersed into the hydrocarbon vapor in a mixing zone 5 resulting in a two-phase vapor 8 laden with a mist of droplets, which is directly transferred to a condensation zone 6 in which both phases precipitate simultaneously. The condensate emulsion 9 thus formed is sent to a separation zone 10 in which the one partial liquid phase 12 of the emulsion mainly consisting of purified hydrocarbon is separated from the other partial liquid phase 15 of the emulsion mainly consisting of solution water with impurities dissolved therein.

### DESCRIPTION

Process and facility for the separation of acid and/or alkaline constituents from hydrocarbons.

The invention relates to a process and to a facility for treating hydrocarbon vapour consisting of at least one aromatic or one olefinic or one paraffinic compound or of a mixture thereof, this hydrocarbon vapour entraining impurities of acid or alkaline nature which consist of at least one water-soluble organic and/or inorganic substance. The product resulting from this process can be obtained with an extremely high purity as regards impurities in the form of organic and inorganic compounds of the elements sulphur, nitrogen, oxygen and chlorine.

For many years, the processing industries have been striving for the complete elimination of impurities from hydrocarbon fractions. This target includes the separation of even minute quantities of impurities of organic and inorganic compounds of the elements sulphur, nitrogen, oxygen and chlorine down to the ppb-range to permit the use of catalyst systems that are more sensitive and selective, especially zeolite-based catalysts. A maximum content of organically bound nitrogen of 30 ppb in benzene, for example, is required for the new catalysts used in the ethyl benzene synthesis, the ppb value being referred to the mass as applies to all of the following ppm and ppb values in the present document.

Impurities as mentioned above are obtained as traces in the ppm range from sulphur, oxygen, nitrogen, chlorine compounds in the product fractions obtained from distillations, absorptions, adsorptions, extractions and extractive distillations. They may result from residues of extraction agents or solvents or their decomposition products or from substances which were contained in the feedstock or were obtained from reactions taking place in the plant.

According to the present state of engineering technology such undesired constituents, provided their reaction is alkaline, are removed from the product by treatment with acid bleaching clay. Such an acid clay treatment, however, involves the following known disadvantages:

Said clay has only a limited lading capacity.

The time of the breakthrough cannot be exactly predicted.

There should always be two clay towers in parallel.

The acid clay cannot be regenerated.

The acid clay is to be steamed after the lading to remove all hydrocarbons.

The steamed clay is to be removed from the tower using the mining technique.

The clay is to be heat-treated to remove any residual hydrocarbons.

After such a treatment, the clay is to be dumped.

According to the present state of the art, impurities that have an acid reaction are removed from the hydrocarbons by means of activated carbon, caustic soda or ion-exchange resins.

Regarding the addition of water to vaporous hydrocarbons, U.S. Pat. No. 4,168,209 provides for the addition of water to a distillation column for extractive distillation above the extraction agent feed point, thus condensing the overhead product and separating the resulting phases. In contrast to the present invention, however, the purpose of adding water is not to remove undesired constituents from the hydrocarbons but to minimise extracting agent losses, primarily within the distillation column into which the water is added. Nor does this U.S. Pat. No. 4,168,209 state what purity could be achieved. Another difference between U.S. Pat. No. 4,168,209 and the present invention is the location of the water feed point: the present invention provides for the water to be added immediately upstream of the condenser and not into an upstream column. The present invention locates the feed point immediately upstream of the condenser and not in an upstream column so that it is possible to use the present invention independently of distillation columns, and, with the purification process carried out according to the present invention, there will be no reflux of water or aqueous solution to a column.

The aim of the process according to the invention is to avoid the disadvantages of the said treatment and the known facilities and to provide a cost-effective process for the treatment of hydrocarbon mixtures that are freed from impurities in the form of organic compounds of the elements sulphur, nitrogen, oxygen and chlorine, i.e. the contents of which may even be in the ppb range. The process according to the present invention serves to purify hydrocarbon vapour consisting of at least one aromatic or olefinic or paraffinic compound or of a mixture thereof, this hydrocarbon vapour entraining impurities of acid or alkaline nature which consist of at least one water-soluble organic and/or inorganic substance.

Both the first claim as well as claim no. 10 which refers to the facility describe the procedure how to reach these aims. The sub-claims 2 to 10 contain further useful embodiments.

In the process according to the present invention, the hydrocarbon feedstock to be purified is evaporated, unless it is already in vaporous phase, for example, as head product of a distillation or stripping column. Superheating is to be avoided or at least to be kept at a minimum to ensure that the major part of the formed hydrocarbon vapour be saturated vapour. In a mixing zone, an aqueous solution is dispersed, for example injected, directly into the hydrocarbon vapour. The amount of solution water referred to the hydrocarbon vapour may range from 1 % by wt. to 20 % by wt., preferably 5 % by wt. The effect will be that at least part of the aqueous solution is evaporated. Heat will be extracted from the hydrocarbon vapour so that part of the hydrocarbons will condense from the vapour phase and mix thoroughly with the droplets of the injected aqueous solution, by which an initial part of the undesired constituents will migrate from the hydrocarbon phase into the aqueous phase where they dissolve more readily in accordance with the ratio of their solubilities.

A two-phase vapour is thus obtained, i.e. a vapour laden with mist of droplets. Its gaseous phase contains the vapours of the hydrocarbon feedstock as well as water vapour. Its liquid phase contains the injected mist of droplets of the solution water with the dissolved impurities. The composition of the liquid phase and the amount of water contained therein depend on the boiling points of the hydrocarbons used. If the hydrocarbon feedstock to be treated consists, for example, mainly of benzene intended for the production of pure benzene, the water content in the mist of droplets will be predominant, as the 100° C. boiling point of the water is above the boiling point of benzene (80.1° C.). If, however, the aqueous solution is injected into a hydrocarbon feedstock to be purified which, for example, mainly consists of toluene, the boiling point of which is 110° C., or mainly of a mixture of ethyl benzene and xylenes, the boiling point of which ranges from 131° C. to 144° C., the two-phase vapour will have a temperature above the boiling point of the water and the hydrocarbon portion in the mist of droplets will predominate. The vapour laden with mist of droplets is directly sent from the mixing zone to a condensation zone. In the condensation zone, the two phases are brought into contact with two cooling surfaces where they jointly condense and transform into a condensed liquor obtained as an emulsion of one liquid in the other liquid.

The two phases thoroughly mix again in the condensation zone so that the remaining portion of the impurities of the hydrocarbon phase can migrate into the aqueous phase in which the remaining impurities dissolve more readily in accordance with the ratio of their solubilities. The condensed liquor formed in the condensation zone consists of a partial liquid phase which mainly contains hydrocarbons and another partial liquid phase which mainly consists of solution water. The condensed liquor is withdrawn from the condensation zone and sent to the separation zone.

In the separation zone, the partial liquid phase containing mainly hydrocarbon is separated from the other partial liquid phase containing mainly aqueous solution with dissolved impurities. The separation of the one partial liquid phase from the other is achieved by making use of their different specific weights, for example, by gravity or centrifugal force or other comparable means. The present invention therefore provides for a water separating device used to remove the one partial liquid phase of the emulsion containing mainly purified hydrocarbon from the other partial liquid phase of the emulsion containing mainly solution water.

The purified hydrocarbon phase is yet to be dried should its further utilisation require that it be anhydrous. The aqueous phase is normally, but not necessarily, split into two part streams. One of these part streams undergoes biological treatment and subsequent disposal. The other part stream is recycled to the injection point and thus constitutes a cycle. The ratio of the two part streams is determined on the basis of the content of dissolved impurities and on the purity of the hydrocarbon product specified for the particular application. The specialist involved will carry out the laboratory tests to this end. If, in individual cases, the max. admissible load is exceeded it may be necessary that only clean water be injected and that the aqueous solution removed in the water separator must to be completely disposed of. Another embodiment of the present invention, therefore, provides for the recycling of at least part of the solution water separated from the emulsion, said part being returned to the mixing zone mentioned above where it is dispersed as part of the aqueous solution and it likewise provides for the withdrawal and disposal of the impurities being entrained in the remaining part of the solution water separated from the emulsion, thus removing said impurities from the solution water cycle.

The beneficial implementation of the process according to this invention is described in more detail using, as an example, an extractive distillation for the production of ultra-pure benzene with the aid of the nitrogen-bearing extraction agent n-formylmorpholine; the use of this process is, of course, not limited to extractive distillations or the removal of n-formylmorpholine or to the purification of benzene.

The extractive distillation process given in the example normally includes two columns, an extractive distillation column and a downstream stripping column. Said columns may also be combined in a divided wall or graduating column. In the first column, i.e. the extractive distillation column, the benzene is washed out from the feed product, which in this case is a benzene fraction, by means of a selective solvent, which in this case is n-formylmorpholine. The non-aromatics are stripped overhead, whereas the benzene and the solvent flow to the bottom of the column. In the second column, which is the stripping column, benzene and solvent are separated. The separated solvent gathers in the bottom of the stripping column and is pumped back to the head of the extractive distillation column for re-use. The benzene leaves the stripping column head in vaporous form. It has been common practice up to now to continue by condensation and collection in the reflux vessel, part of the contents being pumped as reflux to the stripping column. The remaining benzene portion is considered as finished product and piped from the reflux vessel to the battery limit. The average residual content of solvent according to the conventional state of technology is 1 ppm (equal to 1000 ppb) n-formylmorpholine (NFM) or morpholine hydrolysis product, these two substances consisting to about 1/7 of nitrogen, referred to their mass.

The process according to the invention surprisingly permits the reduction of the nitrogen content in the finished product to less than 30 ppb by injecting solution water, preferably with formic acid, these substances being simultaneously injected into the vapour stream of the benzene from the head of the stripping column directly upstream of the condenser. The reason for this phenomenon is that the distribution factor in the ternary system NFM/morpholine-benzene-water which is 30 times higher for NFM/morpholine-water than for NFM/morpholine-benzene. The solubility of water in benzene and that of benzene in water is very low (at 50° C. 1.3 g benzene/1000 g water and 1.56 g water/1000 g benzene). Hence, following a thorough mixing, a phase separation takes place and the NFM solvent is contained in the aqueous phase. The process according to the invention thus has the convincing advantage that the production of an ultra-pure product is feasible by simple means.

Another embodiment of the invention provides for a further increase in the purity of the product beyond the before-mentioned degree if at least part of the aqueous solution dispersed in the mixing zone consists of clean water.

Another embodiment of the invention provides for the pH value of the recycled aqueous solution to be adjusted to a pH-value of slightly above 7, say about 7.5, in order to remove, as salt, the nitrogen compounds already contained in the recycled water from the solution equilibrium. To this end, an acid is admixed to the aqueous solution dispersed in the mixing zone. In the process according to the invention, the acid admixed to the aqueous solution is formic acid.

Another embodiment of the invention provides for the removal of the salts precipitated in the aqueous solution after the addition of acid, using precipitating agents.

Another embodiment of the process according to this invention provides for the admixing of acid being pH controlled.

A specific embodiment of the process according to the invention provides for cooling the aqueous solution before it is dispersed in the mixing zone.

Another embodiment of the process according to the invention provides for the subcooling of the condensate emulsion obtained in the condensation zone before being fed to the separation zone.

The present invention also provides for a facility suited to carry out the process according to this invention. As described above, the process according to the invention provides for dispersing, e.g. by injection, the aqueous solution into the hydrocarbon vapour stream directly upstream of the condenser. It was found that it is particularly effective with regard to the product purity, to combine the mixing zone with the condensation zone in an integral apparatus without any transfer section between the two process steps. Hence, an embodiment of the facility according to the invention provides for a single apparatus in which the mixing and the condensation zones are arranged in a common space enveloping the two zones, the said space being enclosed by the shell of said single apparatus.

The process is also suited for installation in existing plants as the reflux vessels of most fractionation and extraction as well as extractive distillation units are equipped with water separation devices or they can be retrofitted at low cost.

The pure product obtained, which is free from impurities is saturated with water (water in benzene at 50° C.: 1.56 g/1000 g). If the product has to be anhydrous for use in downstream synthesis processes, a distilling or adsorptive drying system can easily be installed downstream.

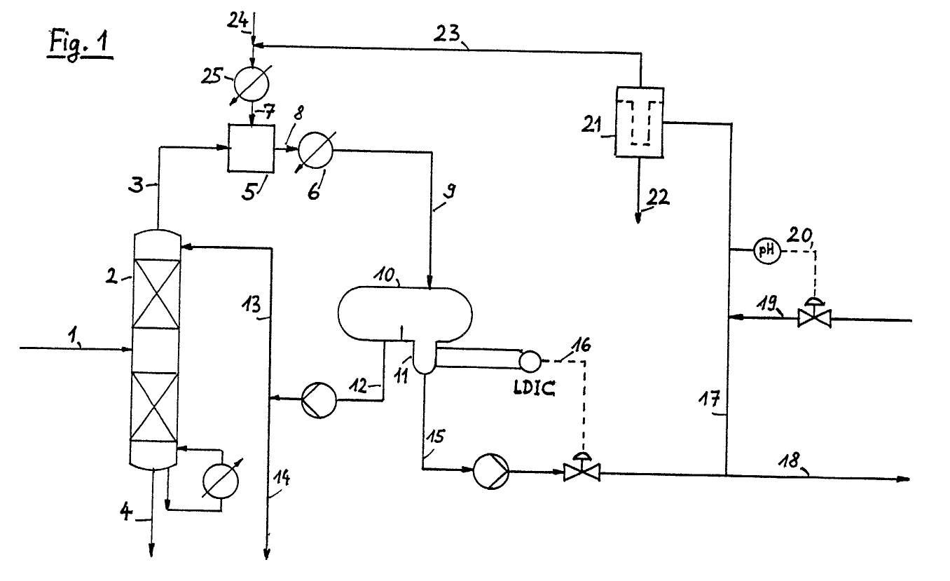

The process according to the invention is represented by a typical example shown in FIG. 1.

The FIGURE shows a plant for the purification of benzene recovered upstream by extractive distillation, the stripping column 2 of which is shown in this diagram. The solvent used is n-formylmorpholine, the residual solvent in the benzene representing the major portion of impurities which must be separated.

Line 1 takes the benzene/solvent mixture from the extractive distillation column to stripping column 2 in which the benzene is separated from the solvent, the benzene being withdrawn as vaporous head product from the stripping column 2 via line 3. The benzene-free solvent is removed from the bottom of stripping column 2 and recycled to the extractive distillation column via line 4.

Immediately before the vaporous benzene enters condenser 6, aqueous solution 7 is injected into mixing zone 5 which is designed as injection device. The aqueous solution 7 is a mixture of deionised water, water vapour condensate, recycled aqueous solution and formic acid. This aqueous solution partly evaporates in mixing zone 5, the energy extracted from the vaporous benzene causing partial condensation of benzene in line 8. In the downstream condenser 6, the evaporated aqueous solution and the residual benzene vapour condense and precipitate, together with the droplets already condensed in condenser 6. Thorough mixing of the benzene and the aqueous solution takes place downstream of the injection point for the aqueous solution in mixing zone 5 as well as in condenser 6. In this process step, the aqueous solution removes most of the impurities from the condensed liquor.

The condensed benzene and the aqueous solution flow via line 9 to reflux vessel 10 which is equipped with a water separation device 11. The purified benzene is withdrawn via line 12, a part stream of which is returned via line 13 to stripping column 2, the remaining part stream being removed as product benzene 14 from the purification unit. The remaining impurities are dissolved in the aqueous solution in reflux vessel 10. The aqueous solution 15 is evacuated from the separation device 11 via a two-phase controller 16, a part stream of it being pumped back via line 17 to the injection point upstream of the condenser. The other part stream of the aqueous solution 15 is transferred as waste water to a biological waste water treatment plant. The ratio of these two streams in lines 17 and 18 is determined on the basis of the content of impurities dissolved in the aqueous solution and the particular benzene purity specified. The solubility equilibria of the impurities for both the aqueous solution phase and the benzene phase must be taken into consideration. The specialist in charge will carry out laboratory tests for this purpose. It may be found in individual cases that only clean water may be injected via line 7 and that aqueous solution 15 separated in reflux vessel 10 has to be completely processed for disposal.

To set the pH-value in a range from 7 to 7.5, formic acid 19 is added in line 17 to the aqueous solution to be used for injection, dosing of the acid being performed via pH controller 20. As a result of reducing the pH value, a solvent salt 22 precipitates and is subsequently removed from the aqueous solution in filter 21. This method prevents any enrichment of the impurities already separated in the aqueous solution. Water from the clean water line 23 is added via line 24 to the aqueous solution to make up for the cycle water that leaves the purification unit via line 1, either dissolved in the product benzene 14 or as waste water. To intensify the condensation effect in mixing zone 5, the aqueous solution can be cooled in water cooler 25 if required.

1. Process for the purification of hydrocarbon vapour consisting of at least one aromatic or one olefinic or one paraffinic compound or of a mixture thereof, said hydrocarbon vapour entraining impurities of acid or alkaline nature which consist of at least one water-soluble organic and/or inorganic substance,
characterised in that an aqueous solution is dispersed in a mixing zone and thus added to the hydrocarbon vapour, resulting in a two-phase vapour laden with a mist of droplets which is directly transferred to a condensation zone in which both phases precipitate simultaneously, the condensate emulsion thus formed being sent to a separation zone, in which the one partial liquid phase of the emulsion mainly consisting of purified hydrocarbon is separated from the other partial liquid phase of the emulsion mainly consisting of solution water with the impurities dissolved therein.

2. Process according to claim 1, characterised in that the separation of the one partial liquid phase of the emulsion mainly consisting of purified hydrocarbon from the other partial liquid phase of the emulsion mainly consisting of solution water is accomplished by means of a water separating device.

3. Process according to any one of the preceding claims 1 to 2, characterised in that at least part of the solution water separated from the emulsion is recycled and then dispersed as part of the aqueous solution in the mixing zone and in that the impurities are removed from the solution water cycle with the remaining part of the solution water separated from the emulsion and sent to the disposal facilities.

4. Process according to any of the preceding claims 1 to 3, characterised in that at least part of the aqueous solution dispersed in the mixing zone consists of clean water.

5. Process according to any of the preceding claims 1 to 4, characterised in that an acid is admixed to the aqueous solution dispersed into the mixing zone.

6. Process according to claim 5 above, characterised in that the acid admixed to the aqueous solution is formic acid.

7. Process according to any one of claims 5 or 6, characterised in that the salts precipitated after admixing acid to the aqueous solution are removed from the aqueous solution with the aid of precipitants.

8. Process according to any of the preceding claims 5 to 7, characterised in that the acid admixture is pH controlled.

9. Process according to any of the preceding claims 1 to 8, characterised in that the aqueous solution is cooled prior to being dispersed into the mixing zone.

10. Process according to any of the preceding claims 1 to 9, characterised in that the condensate emulsion that forms in the condensation zone is submitted to subcooling prior to entering the separation zone.

11. Facility for the performance of the process according to at least one of the preceding claims 1 to 10, characterised in that the mixing zone and the condensation zone a recombined in a common space enveloping the two zones and that this space is enclosed by the shell of a single apparatus.
